(19) United States
(12) Patent Application Publication
(10) Pub. No.: US 2005/0136083 A1
(43) Pub. Date: Jun. 23, 2005

(54) Cosmetic article with improved skin penetration comprising simultaneous application of an active ingredient and use of an abrasive device

(75) Inventors: Franz Stab, Echem (DE); Thomas Blatt, Wedel (DE); Melanie Schmidt, Hamburg (DE); Holger Lenz, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/993,913

(22) Filed: Nov. 19, 2004

(30) Foreign Application Priority Data
Nov. 26, 2003 (DE) 103 55 229.4

(51) Int. Cl. A61K 7/00; A61K 9/70; A61K 31/198; A61K 31/12

(52) U.S. Cl. 424/401; 424/443; 514/690; 514/565

(57) ABSTRACT

The invention is a method for improving the penetration of a cosmetic or dermatological preparation into the skin by simultaneously applying the preparation to the skin and using a device to remove the skin. The present invention also includes an article that includes both the device and a cosmetic or dermatological preparation and a kit including these components and instructions for their simultaneous use.

### FIELD OF THE INVENTION

The present invention relates to a cosmetic article with improved penetration of an active ingredient that includes the simultaneous use of an abrasive device.

### BACKGROUND OF THE INVENTION

The specification WO 94/18879 discloses devices for the cosmetic peeling of the skin using a more specifically determined fabric.

The specifications U.S. Pat. No. 4,459,987 and U.S. Pat. No. 4,712,552 disclose flexible cosmetic emery sheets based on certain polymers which carry emery particles on at least one side.

The specification EP 1219254 discloses an apparatus for the removal of skin.

In none of these documents is anything disclosed concerning applications of such devices extending beyond mere peeling of the skin.

### SUMMARY OF THE INVENTION

It has proven unforeseeable by the person skilled in the art that a cosmetic and/or dermatological product comprising a device for the removal of skin and an active ingredient-containing cosmetic and/or dermatological preparation where, during use of the product, the device is employed and the preparation is dosed at the same time, overcome the shortcomings of the prior art. When using such a product, the mechanical irritation acts as a physiological stimulus of the skin, which is intensified or compensated for through simultaneous use of the active ingredient.

It has also been found that it is preferred if the device is a large-pored face glove. By using this, the skin is firstly cleansed of dirt. The effect described above takes place to a greater degree.

The large-pored face glove used may be a sisal glove, a peeling glove made of luffa sponge or brush devices supplied with bobbles in glove form or other comparable devices.

It is particularly preferred if the preparation is in the form of a W/O cream. In this connection, it is particularly preferred if an active ingredient is present in the preparation which is chosen very particularly preferably from the group of antioxidants. It is very extraordinarily preferred if it is ubiquinone, ubiquinol, creatine and/or creatinine.

For all this it is preferred if the preparation is dosed by the device as it is used. The invention also covers the use of such a device for increasing the penetration of a cosmetic and/or dermatological active ingredient applied over the course of 30 s, particularly preferably for improving the energy metabolism and/or for stimulating collagen neogenesis or further dermal components. Moreover, the invention also covers a non-therapeutic method for the treatment and prophylaxis of degenerative skin symptoms by using one of the products described above.

Preferably, the abrasive device to be used as cosmetic article simultaneously contains, for the purposes of the invention, active ingredients whose use concentration is in the range from 0.0001% to 50% by weight, particularly preferably 0.01% to 10% by weight (in each case based on the total weight of the preparation).

Use of the device used according to the invention simultaneously with a cosmetic or topical dermatological preparation surprisingly enables effective treatment, but also prophylaxis of certain degenerative symptoms of the skin (e.g. skin slackening, wrinkles, lachrymal sacs, age spots, telangiectases, disturbance of the osmolyte balance, of the natural skin pH, and disappearance of the epidermal and dermal cell layers, the constituents of connective tissue, the retinal cones and capillary vessels of the skin) and/or of the skin appendages,
of environmentally induced (caused by smoking, smog, reactive oxygen species, free radicals and the like) negative changes in the skin and the skin appendages,
of deficient, sensitive or hypoactive skin conditions or deficient, sensitive or hypoactive conditions of skin appendages,
for reduced skin thickness, dermotensions
for skin slackening, skin fatigue, stretch marks
of pigmentation disorders,
of horny layer barrier disorders,
for changes in the transepidermal water loss and the normal moisture content of the skin,
for changes in the ceramide, lipid and energy metabolism of healthy skin,
of hair loss and for improved hair growth,
for deviations from the normal cell-cell communication in the skin,
for changes in the normal fibroblast and keratinocyte proliferation,
for changes in the normal fibroblast and keratinocyte differentiation, and
of inflammatory skin conditions, and atopic eczema, seborrhoeic eczema, polymorphous photodermatosis, psoriasis, and vitiligo.

The device according to the invention together with active ingredient, however, are also surprisingly effective
for calming sensitive or irritated skin,
for maintaining normal collagen, hyaluronic acid, elastin and glycosaminoglycan homeostasis,
for stimulating oestrogen synthetase,
for increased activation of proteolytic enzymes in the skin, such as, for example, metalloproteinases,
for stimulating intracellular DNA synthesis, in particular in deficient or hypoactive skin conditions,
for wound healing disorders,
for increasing cell renewal and regeneration of the skin,
for reducing the production of sebum,
for preventing the formation and for the removal of comedones and/or for establishing normal sebum homeostasis,
for preventing or overcoming seborrhoeic symptoms,
for increasing the skin's own protective and repair mechanisms (for example for dysfunctional enzymes, DNA, lipids, proteins), and
for the pre- and post-treatment in cases of topical application of laser and abrasive treatments, which serve, for example, to reduce skin wrinkles and scars, to counteract the resulting skin irritations and to promote the regeneration processes in the damaged skin.

The omission of an individual constituent can affect the unique properties of the overall composition. Accordingly, the stated constituents of the preparations according to the invention are preferably included to carry out the invention.

### DETAILED DESCRIPTION OF THE INVENTION

Degenerative skin symptoms for the purposes of the present specification are wrinkles, slackening, degeneration, age spots, pigment disorders, circulatory disorders, itching, stress sensitivity, dry skin, cellulite, dermotensions, and fatigue states.

Devices for the removal of skin for the purposes of the present specification are sponges, woven or nonwoven textiles supplied with abrasive particles, textiles containing abrasive fibres or particles, such as wash gloves with or without coconut or hemp fibres, sea cucumber skeleton (luffa), and coarsely woven textiles.

Abrasive particles for the purposes of the present specification are, for example, quartz sand, almond bran, polyethylene, aluminium trioxide, ground fruit cores (walnut or apricot shells), salt crystals, jojoba leaves, ground sepia shells, pumice, zeolites, and silicon dioxide.

For the purposes of the present specification, sponges are natural or artificial. Natural sponges are, for example, coral sponges or pumice sponges, artificial sponges are, for example, large-pored viscose sponges, velour sponges, polyester sponges or polyether sponges. They have a mostly irregular cell structure and pores of varying size. They are flexible and elastic.

Textiles for the purposes of the present specification are wipes, nonwovens, felt, knits, wovens, pads, but also cords, strings, ropes and threads.

Abrasive fibres for the purposes of the present specification are natural or synthetic fibres which have abrasive properties as a result of the nature of their surface, such as hardness or structure, or as a result of their dimensions, such as, for example, hemp, sisal, and coconut fibres.

For the purposes of the present specification, “simultaneously” also means with slight time delay. In particular, for example two functions or properties of a described product simultaneous use of means that these both play a part during one application operation of a product. This can be achieved either by both functions or properties playing a part one after the other, possibly by them being physical separable or separate prior to use. In this case, simultaneously covers a period from 0 to 30 seconds. Preferably, functions or properties play a part simultaneously by them being arranged in an integrated manner in an article. In this case too, “simultaneously” covers a time interval from 0 to about 5 seconds if, for example, the sites of the functions or properties are arranged at a distance from one another, for example 20 mm, and are activated during use at a certain rate, for example 50 mm/s along an application path.

Emulsions are generally understood as meaning heterogeneous systems which consist of two liquids which are immiscible or have only limited miscibility with one another and which are usually referred to as phases. In an emulsion, one of the two liquids is dispersed in the form of very fine droplets in the other liquid.

If the two liquids are water and oil and oil droplets are present in finely divided form in water, then this is an oil-in-water emulsion (O/W emulsion, e.g. milk). The basic character of an O/W emulsion is determined by the water. In the case of a water-in-oil emulsion (W/O emulsion, e.g. butter), the principle is reversed, the basic character here being determined by the oil.

Antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, y-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulphoximine compounds (e.g. buthionine sulphoximines, homocysteine sulphoximine, buthionine sulphones, penta-, hexa-, heptathionine sulphoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, maleic acid), humic acid, bile acid, bile extracts, bilirubin, biliveridin, EDTA, EGTA and derivatives thereof, unsaturated fatty acids and derivatives thereof (e.g. γ-linolenic acid, linoleic acid, oleic acid), isoflavonoids, licochalcones, licochalcones A (in particular from Radix Glycyrrhizae inflatae), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and also coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these specified active ingredients which are suitable according to the invention.

The amount of the abovementioned antioxidants (one or more compounds) in the preparations according to the invention is preferably 0.001 to 30% by weight, particularly preferably 0.05-20% by weight, in particular 1-10% by weight, based on the total weight of the preparation.

If vitamin E and/or derivatives thereof are the antioxidant or antioxidants, it is advantageous to choose their particular concentrations from the range 0.001-10% by weight, based on the total weight of the formulation.

If vitamin A or vitamin A derivatives, or carotenes or derivatives thereof are the antioxidant or the antioxidants, it is advantageous to choose their particular concentration from the range 0.001-10% by weight, based on the total weight of the formulation.

Besides said ingredients, preparations according to the invention can comprise oils, waxes, lipids, emulsifiers, thickeners, skin moisturizers, UV filters, complexing agents, preservatives, perfume, powder raw materials, pigments, dyes.

For all this it is in individual cases possible that the above-mentioned concentrations given are slightly exceeded or not reached and nevertheless preparations according to the invention are obtained. In view of the widely scattered diversity of suitable components of such preparations, this comes as no surprise to the person skilled in the art, meaning that he knows that such excesses or shortfalls do not depart from the essence of the present invention.

The examples below serve to illustrate the present invention without limiting it. The numerical values in the examples are percentages by weight, based on the total weight of the particular preparations.

### EXAMPLES

The coconut rope glove used is a standard commercial product, e.g. from Acca Kappa. A luffa sponge can also be used as massage belt and as large-pored make-up removal pad (e.g. from Wellsana).

For use, the skin surface to be treated is rubbed with the clean, dry or slightly moistened sponge a few times, for example 3× with slight pressure, for example with circular movements, similarly to washing the body with a flannel. The preparation is then rubbed into the surface of the skin. This is carried out like the normal application of a cream or lotion.

### Example 1

### Application of a Gel Cream After Treating the Skin with a Luffa Sponge

| | Mass content (%) |
|---|---|
| Acrylates/C10-30 alkyl acrylate | 0.40 |
| crosspolymer | |
| Carbomer | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclomethicone | 5.00 |
| Dimethicone | 1.00 |
| Creatine | 0.20 |
| Glycerol | 3.00 |
| Sodium hydroxide | q.s. |
| Preservative | q.s. |
| Perfume | q.s. |
| Water, demineralized | ad 100.0 |
| pH adjusted to 6.0 | |

### Example 2

### Application of a W/O Cream Following Treatment of the Skin with a Large-Pored Make-Up Removal Pad

| | Mass content (%) |
|---|---|
| Lameform TGI | 3.50 |
| Glycerol | 3.00 |
| Dehymuls PGPH | 3.50 |
| Ubiquinone | 0.03 |
| Preservative | q.s. |
| Perfume | q.s. |
| Water, demin. | ad 100.0 |
| Magnesium sulphate | 0.6 |
| Isopropyl stearate | 2.0 |
| Caprylyl ether | 8.0 |
| Cetearyl isononanoate | 6.0 |

### Example 3

### Application of a W/O/W Cream Following Treatment of the Skin with a Coconut Rope Glove

| | Mass content (%) |
|---|---|
| Glyceryl stearate | 3.00 |
| PEG-100 stearate | 0.75 |
| Behenyl alcohol | 2.00 |
| Caprylic/capric triglyceride | 8.0 |
| Octyldodecanol | 5.00 |
| C12-15 alkyl benzoate | 3.00 |
| Taurine | 1.00 |
| Magnesium sulphate (MgSO₄) | 0.80 |
| EDTA | 0.10 |
| Preservative | q.s. |
| Perfume | q.s. |
| Water, demineralized | ad 100.0 |
| pH adjusted to 6.0 | |

1. A method for treating the skin with a cosmetic or dermatological preparation, comprising the step of simultaneously applying a cosmetic or dermatological preparation to the skin and using a device to remove the skin.

2. The method according to claim 1, wherein the device is a large-pored face glove.

3. The method according to claim 1, wherein the preparation is a water-in-oil cream.

4. The method according to claim 1, wherein the preparation includes an active ingredient.

5. The method according to claim 4, wherein the active ingredient is selected from the group consisting of antioxidants.

6. The method according to claim 4, wherein the active ingredient is selected from the group consisting of ubiquinone and ubiquinol.

7. The method according to claim 4, wherein the active ingredient is selected from the group consisting of creatine and creatinine.

8. The method according to claim 1, wherein the device doses the preparation to the skin while the device removes the skin.

9. The method according to claim 1, wherein the cosmetic or dermatological active ingredient is applied over the course of 30 s and the device increases the penetration of the active ingredient.

10. A method for improving energy metabolism, comprising the step of simultaneously applying a cosmetic or dermatological preparation to the skin and using a device to remove the skin.

11. A method for stimulating collagen neogenesis, comprising the step of simultaneously applying a cosmetic or dermatological preparation to the skin and using a device to remove the skin.

12. A non-therapeutic method for the treatment or prophylaxis of degenerative skin symptoms, comprising the step of simultaneously applying a cosmetic or dermatological preparation to the skin and using a device to remove the skin.

13. A cosmetic or dermatological article, comprising a device for the removal of skin and an active ingredient-containing cosmetic or dermatological preparation, said device capable of being used simultaneous with the application of the preparation to the skin.

14. The article according to claim 13, wherein the device is capable of supplying the preparation to the skin as the device removes the skin.

15. A kit, comprising:
a device for the removal of skin;
a cosmetic or dermatological preparation comprising an active ingredient;
said device capable of being used simultaneous with the application of the preparation to the skin.

16. The kit according to claim 15, wherein the device is capable of supplying the preparation to the skin as the device removes the skin.

17. The kit according to claim 15, further comprising instructions for the simultaneous operation of the device and application of the preparation to the skin.
